(19) United States
(12) Patent Application Publication
(10) Pub. No.: US 2003/0069307 A1
(43) Pub. Date: Apr. 10, 2003

(54) 2-(3,4-Dihydroxyphenyl)ethyl-substituted carbonic acid derivatives and their use

(76) Inventor: Jakob Peter Ley, Holzminden (DE)

Correspondence Address: BAYER CORPORATION, PATENT DEPARTMENT, 100 BAYER ROAD, PITTSBURGH, PA, 15205, US

(21) Appl. No.: 10/219,568

(22) Filed: Aug. 15, 2002

(30) Foreign Application Priority Data
Aug. 17, 2001 (DE) 10140443.3

(51) Int. Cl. A61K 31/265; C07C 69/96; A61K 47/00; C07C 329/06; C07C 329/14

(52) U.S. Cl. 514/512; 558/275; 558/245; 558/230; 424/439

(57) ABSTRACT

The invention relates to the use of 2-(3,4-dihydroxyphenyl)ethyl-substituted carbonic acid derivatives as antioxidants or free-radical scavengers, in particular to the use for protecting human cells and tissues from the harmful, aging-accelerating effects of free-radicals and reactive oxygen compounds.

### FIELD OF THE INVENTION

The present invention relates to the use of 2-(3,4-dihydroxy-phenyl)ethyl-substituted carbonic acid derivatives as antioxidants or free-radical scavengers, in particular the use for protecting human cells and tissues from the harmful effects of free radicals and reactive oxygen compounds. The present invention further relates to cosmetic preparations, dermatological preparations or preparations serving for nutrition or pleasure comprising these 2-(3,4-dihydroxy-phenyl)ethyl-substituted carbonic acid derivatives.

### BACKGROUND OF THE INVENTION

Antioxidants are substances, which at low concentrations compared with the oxidizable substrate, significantly delay or entirely inhibit oxidation. Many antioxidants simultaneously function as free-radical scavengers and/or as complexing agents for heavy metal ions.

It is worthwhile finding substances which, in physiological systems, support the natural defense mechanisms against free radicals and reactive oxygen compounds or, as protective substances in cosmetics, pharmaceuticals and in foods and drinks protect their oxidation-sensitive constituents against autoxidation.

### SUMMARY OF THE INVENTION

It is an object of the present invention to develop antioxidants having strong specific free-radical scavenging and/or antioxidant action for use in cosmetic and pharmaceutical preparations and in foods and drinks and for protecting mammalian cells and tissue.

Therefore, the present invention relates to the use of 2-(3,4-dihydroxyphenyl)ethyl-substituted carbonic acid derivatives of the general formula I

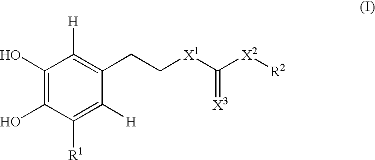

wherein

R¹ is a hydrogen atom, a lower alkyl, lower alkenyl, 1-oxo lower alkyl or 1-oxo lower alkenyl group or a group —O—R³, where R³ is a hydrogen atom, a lower alkyl, a lower alkenyl, 1-oxo lower alkyl or 1-oxo lower alkenyl group, and

X¹, X² and X³ independently of one another are oxygen, sulfur or NH, and

R² is a branched or unbranched cyclic or extended alkyl group having 1 to 22 carbon atoms or a branched or unbranched cyclic or extended alkenyl group having 2 to 22 carbon atoms, in which one or more of the carbon atoms can be replaced by oxygen atoms, or a nuclear-substituted arylalkyl group having 7 to 15 carbon atoms,

as antioxidants or free-radical scavengers.

### DETAILED DESCRIPTION OF THE INVENTION

A lower alkyl group preferably contains 1 to 5 carbon atoms and can be, for example: methyl, ethyl, 1-propyl, 2-propyl, 1-butyl, 2-butyl, tert-butyl, 2-methylprop-1-yl, cyclopropyl, cyclopropylmethyl, 2,2-dimethylcyclopropyl, cyclobutyl, 1-, 2- or 3-pentyl-, 2-methylbut-1-yl, 2-methylbut-2-yl, 3-methylbut-1-yl, 3-methylbut-2-yl or cyclopentyl.

A lower alkenyl group preferably contains 2 to 5 carbon atoms and can be, for example: ethenyl, prop-2-en-1-yl, prop-1-en-1-yl, prop-1-en-2-yl, 1- or 2-cyclopropenyl-, but-1-en-1-yl, but-1-en-2-yl, but-1-en-3-yl, but-2-en-1-yl, but-3-en-1-yl, but-2-en-2-yl, 2-methylprop-1-en-1-yl, 2-methylprop-2-en-1-yl, 1,3-butadien-1-yl, 1,3-butadien-2-yl, pent-1-en-1-yl, pent-1-en-2-yl, pent-1-en-3-yl, pent-1-en-4-yl, pent-2-en-1-yl, pent-2-en-2-yl, pent-2-en-3-yl, pent-2-en-4-yl, pent-3-en-1-yl, pent-4-en-1-yl, 1,3-pentadien-1-yl, 1,3-pentadien-2-yl, 1,3-pentadien-3-yl, 2,4-pentadien-2-yl, 2,4-pentadien-1-yl, 1,4-pentadien-1-yl, 1,4-pentadien-2-yl, 1,4-pentadien-3-yl, 1-, 2- or 3-cyclopentenyl, 1-, 2- or 3-cyclopentadienyl, 3-methylbut-1-en-1-yl, 3-methylbut-1-en-2-yl, 3-methylbut-1-en-3-yl, 3-methylbut-1-en-4-yl, 3-methylbut-2-en-1-yl, 3-methylbut-2-en-2-yl, 3-methylbut-2-en-4-yl, 2-methylbut-1-en-1-yl, 2-methylbut-1-en-3-yl, 2-methylbut-1-en-4-yl, 2-methylidenebut-1-yl, 2-methyl-1,3-butadien-1-yl, 2-methyl-1,3-butadien-3-yl, 2-methyl-1,3-butadien-4-yl, 2-methylidenebut-3-en-1-yl and where appropriate, the respective possible Z- and E-isomers of the above-mentioned radicals.

Preferably, a 1-oxo lower alkyl group contains 1 to 5 carbon atoms and can be, for example: formyl, acetyl, propanoyl, butanoyl, 2-methyl-propanoyl, pentanoyl, 2- or 3-methylbutanoyl, 2,2-dimethylpropanoyl or cyclopropylcarboxyl.

A 1-oxoalkenyl group can preferably contain 3 to 5 carbon atoms and can be, for example: prop-2-enoyl, 2-methylprop-2-enoyl, 2-ethylprop-2-enoyl, E- or Z-2-butenoyl, 3-butenoyl, E- or Z-2-methylbut-2-enoyl, E- or Z-3-methylbut-2-enoyl, Z- or E-2-pentenoyl, Z- or E-3-pentenoyl.

An unbranched, branched or cyclic alkyl group can contain 1 to 22, preferably 1 to 20, more preferably 1 to 18 carbon atoms. Those which may be mentioned by way of example are: methyl, ethyl, 1-propyl, 2-propyl, 1-butyl, 2-butyl, tert-butyl, 2-methyl, 2-methylprop-1-yl, cyclopropyl, cyclopropylmethyl, 2,2-dimethylcyclopropyl, cyclobutyl, cyclopentyl, cyclohexyl and various positional isomers of methylpentyl, menthyl, 1-pentyl, 1-hexyl, 1-heptyl, 1-octyl, 2-ethylhexyl, 1-nonyl, 1-decyl, 1-undecyl, 1-dodecyl, 1-tridecyl, 1-tetradecyl, 1-pentadecyl, 1-hexadecyl, 1-heptadecyl and 1-octadecyl.

An unbranched, branched or cyclic alkenyl group can contain 2 to 22, preferably 2 to 20, more preferably 2 to 18 carbon atoms. Those which may be mentioned by way of example are: ethenyl, prop-2-en-1-yl, prop-1-en-1-yl, prop-1-en-2-yl, 1- or 2-cyclopropenyl-, but-1-en-1-yl, but-1-en-2-yl, but-1-en-3-yl, but-2-en-1-yl, but-3-en-1-yl, but-2-en-2-yl, 2-methylprop-1-en-1-yl, 2-methylprop-2-en-1-yl, 1,3-butadien-1-yl, 1,3-butadien-2-yl, pent-1-en-1-yl, pent-1-en-2-yl, pent-1-en-3-yl, pent-1-en-4-yl, pent-2-en-1-yl, pent-2-en-2-yl, pent-2-en-3-yl, pent-2-en-4-yl, pent-3-en-1-yl, pent-4-en-1-yl, 1,3-pentadien-1-yl, 1,3-pentadien-2-yl, 1,3-pentadien-3-yl, 2,4-pentadien-2-yl, 2,4-pentadien-1-yl, 1,4-pentadien-1-yl, 1,4-pentadien-2-yl, 1,4-pentadien-3-yl, 1-, 2- or 3-cyclopentenyl, 1-, 2- or 3-cyclopentadienyl, 3-methylbut-1-en-1-yl, 3-methylbut-1-en-2-yl, 3-methylbut-1-en-3-yl, 3-methylbut-1-en-4-yl, 3-methylbut-2-en-1-yl, 3-methylbut-2-en-2-yl, 3-methylbut-2-en-4-yl, 2-methylbut-1-en-1-yl, 2-methylbut-1-en-3-yl, 2-methylbut-1-en-4-yl, 2-methylidenebut-1-yl, 2-methyl-1,3-butadien-1-yl, 2-methyl-1,3-butadien-3-yl, 2-methyl-1,3-butadien-4-yl, 2-methylideneebut-3-en-1-yl, the various positional isomers and double-bond isomers of the heptenyl, octenyl, nonenyl, decenyl, dodecenyl, tetradecenyl, hexadecenyl and octadecenyl radical, Z-hexadeca-9-en-1-yl, Z-octadeca-9-en-1-yl, Z,Z-octadeca-9,12-dien-1-yl, Z,Z,Z-octadeca-9,12,15-trien-1-yl and E-octadeca-9-en-1-yl.

A nuclear-substituted arylalkyl group can contain 7 to 15 carbon atoms, preferably 7 to 8 carbon atoms, with the proviso that at least one further substituent on the aromatic part is not hydrogen. In particular, preference is given to a nuclear-substituted benzyl, a 2- or 1-phenylethyl group.

Preferred substituents of the nuclear-substituted arylalkyl group are: hydrogen atoms, lower alkyl, hydroxyl, lower alkoxy, thio, lower alkylthio, amino, lower alkylamino, di-lower-alkylamino, phosphate, lower alkyl-phosphate, di-lower-alkylphosphate, sulfonic acid, lower alkylsulfonate, sulfonamide, di-lower-alkylsulfonamide or lower alkylsulfonamide radicals. In particular, preference is given to hydrogen atoms, lower alkyl, hydroxyl and lower alkoxy radicals.

Preference is given to the use of 2-(3,4-dihydroxyphenyl)ethyl-substituted carbonic acid derivatives of the general formula 1,

wherein

R¹ is a hydrogen atom, a lower alkyl group having 1 to 5 carbon atoms or a group —O—R³ where R³ is a hydrogen atom or a methyl group, and

X¹, X² and X³ independently of one another are oxygen, sulfur or NH, and

R² is a branched or unbranched, cyclic or extended alkyl group having 1 to 18 carbon atoms or a branched or unbranched cyclic or extended alkenyl group having 2 to 20 carbon atoms, or a nuclear-substituted benzyl, nuclear-substituted 2-phenylethyl or nuclear-substituted 1-phenylethyl radical,

as antioxidants or free-radical scavengers.

In particular, preference is given to the use of 2-(3,4-dihydroxyphenyl)ethyl-substituted carbonic acid derivatives of the general formula 1,

wherein

R¹ is a hydrogen atom, a methoxy or hydroxyl group, and

X¹ is a group NH, and

X² and X³ independently of one another are oxygen, sulfur or NH, and

R² is a branched or unbranched cyclic or extended alkyl group having 1 to 18 carbon atoms or a branched or unbranched cyclic or extended alkenyl group having 2 to 18 carbon atoms,

as antioxidants or free-radical scavengers.

Individual inventive compounds which may be mentioned by way of example are:

N-[2-(3,4-dihydroxyphenyl)ethyl]-O-methylurethane

N-[2-(3,4-dihydroxyphenyl)ethyl]-O-[(1R,3R,4S)-menthyl]urethane

N-[2-(3,4-dihydroxyphenyl)ethyl]-O-hexylurethane

N-[2-(3,4-dihydroxyphenyl)ethyl]-O-(2-ethylhexyl)urethane

N-[2-(3,4-dihydroxyphenyl)ethyl]-N′-hexylthiourea

N-[2-(3,4-dihydroxyphenyl)ethyl]-N′-hexylurea.

If, in the inventive 2-(3,4-dihydroxyphenyl)ethyl-substituted carbonic acid derivatives of the general formula 1, at least one of the radicals X¹, X² and X³ is NH, inventive compounds can also be present in the form of their tautomers.

Surprisingly, it has now been found that the inventive 2-(3,4-dihydroxyphenyl)ethyl-substituted carbonic acid derivatives are very good free-radical scavengers and particularly strong antioxidants. In particular, the inventive 2-(3,4-dihydroxyphenyl)ethyl-substituted carbonic acid derivatives are able to suppress the harmful effects of free radicals and/or oxidative processes in or on the skin and to support the natural antioxidative processes.

The inventive 2-(3,4-dihydroxyphenyl)ethyl-substituted carbonic acid derivatives are, moreover, very good antioxidants for highly unsaturated lipids such as, for example, squalene, lycopene, carotenes, docosahexaenoic acid, eicosapentaenoic acid, α- or β-linolenic acid or linoleic acid, and for fatty oils containing (poly)unsaturated fatty acids, for example soya bean oil, linseed oil, thistle oil, borage seed oil, evening primrose oil, fish oil, olive oil, blackcurrant seed oil or sunflower seed oil. Therefore, they can be used as antioxidants in preparations which comprise such lipids and are used in nutrition or for pleasure. In particular, the inventive 2-(3,4-dihydroxyphenyl)ethyl-substituted carbonic acid derivatives are also outstandingly suitable for stabilizing pure lipids or fatty oils or mixtures of the same.

The inventive 2-(3,4-dihydroxyphenyl)ethyl-substituted carbonic acid derivatives reinforce in physiological systems, the natural defense mechanisms against free radicals and reactive oxygen compounds and, in cosmetics, pharmaceuticals and foods and drinks, protect their oxidation-sensitive constituents from autoxidation or photooxidation.

The inventive 2-(3,4-dihydroxyphenyl)ethyl-substituted carbonic acid derivatives can therefore, be used in cosmetic preparations, dermatological preparations and preparations serving for nutrition or pleasure for protecting cells and tissues of mammals, in particular humans, from the harmful effects of free radicals and reactive oxygen species (oxidation and photooxidation). Obviously, the inventive 3,4-dihydroxybenzyl-substituted carbonic acid derivatives can also be used in other compositions, for example pharmaceutical compositions or preparations, preparations for protecting or affecting plants, preparations for supplementing foods, in preparations for producing foods and drinks or in other products, for example oxidation-sensitive natural or synthetic polymers (for example rubber, polyolefins), as antioxidants or free-radical scavengers.

The amount of the inventive 2-(3,4-dihydroxyphenyl)ethyl-substituted carbonic acid derivatives in the inventive preparations is 0.0001% by weight to 10% by weight, preferably 0.001 to 5% by weight, more preferably 0.001% by weight to 1% by weight, based on the total weight of the compositions.

Some of the 2-(3,4-dihydroxyphenyl)ethyl-substituted carbonic acid derivatives are known. Thus, JP 60/115,558 describes the synthesis of N-2-(3,4-dihydroxyphenyl)ethyl-O-n-methylurethane and N-2-(3,4-dihydroxy-phenyl)ethyl-O-n-hexylurethane, and in New J. Chem. 1998, vol. 22, issue 2, pp. 129-135, the synthesis of N,N′-bis-[2-(3,4-dihydroxy-phenyl)ethyl]urea is described. The inventive 2-(3,4-dihydroxyphenyl)ethyl-substituted carbonic acid derivatives, however, have not been described either in connection with an antioxidant and/or free-radical scavenging action nor in connection with their use in cosmetic applications, foods or drinks.

2-(3,4-Dihydroxyphenyl)ethyl-substituted carbonic acid derivatives of the general formula (II)

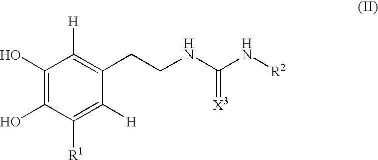

wherein

R¹ is a hydrogen atom, a lower alkyl, lower alkenyl, 1-oxo lower alkyl or 1-oxo lower alkenyl group or a group —O—R³, where R³ is a hydrogen atom, a lower alkyl, lower alkenyl, 1-oxo lower alkyl or 1-oxo lower alkenyl group, and

X³ is oxygen, sulfur or NH, and

R² is a branched or unbranched cyclic or extended alkyl group having 1 to 22 carbon atoms or a branched or unbranched cyclic or extended alkenyl group having 2 to 22 carbon atoms,

are novel.

Preference is given to 2-(3,4-dihydroxyphenyl)ethyl-substituted carbonic acid derivatives of the general formula (II),

where

R¹ is a hydrogen atom, a lower alkyl group having 1 to 5 carbon atoms or a group —O—R³ where R³ is a hydrogen atom or a methyl group, and

X³ is oxygen or sulfur, and

R² is a branched or unbranched cyclic or extended alkyl group having 1 to 18 carbon atoms or a branched or unbranched cyclic or extended alkenyl group having 2 to 20 carbon atoms.

Inventive individual compounds which may be mentioned by way of example are:

N-[2-(3,4-dihydroxyphenyl)ethyl]-N′-hexylthiourea

N-[2-(3,4-dihydroxyphenyl)ethyl]-N′-hexylurea.

The 2-(3,4-dihydroxyphenyl)ethyl-substituted carbonic acid derivatives of the general formula (II) can be synthesized by a process in which 2-(3,4-dihydroxyphenyl)ethylamines of the general formula (III)

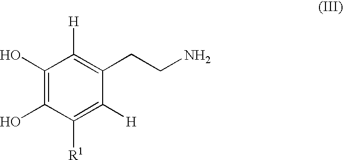

where R¹ has the meaning defined above,

or cationic salts thereof

are reacted with a heterocumulene of the general formula (IV),

X⁴═C═N—R² (IV)

where

X⁴ is an oxygen atom or a sulfur atom and

R² has the meaning specified above, or

with phosgene, thiophosgene or triphosgene with or without solvent and with or without the addition of an auxiliary base and then either directly after purification or without purification with a compound of the general formula (V),

Z-R² (V)

where

Z is a group —O—H, —S—H, —NH₂ or —(NH₃)⁺ and

R² has the meaning specified above

with or without solvent and with or without addition of an auxiliary base.

The 2-(3,4-dihydroxyphenyl)ethyl-substituted carbonic acid derivatives of the general formula (II) can preferably be obtained by reacting 2-(3,4-dihydroxyphenyl)ethylamines of the general formula (III), where R¹ has the meaning specified above, with a heterocumulene of the general formula (IV), where R² has the meaning specified above, in a solvent or solvent mixture, preferably selected from the group containing water, acetone, 1,4-dioxane, tetrahydrofuran, aliphatic esters of aliphatic alcohols (for example ethyl acetate), chlorinated solvents (for example chloroform) or aromatic solvents (for example benzene), and advantageously with addition of one or more auxiliary bases, preferably selected from the group containing the alkali metal hydroxides (for example, NaOH), alkali metal carbonates (for example Na₂CO₃ or NaHCO₃), the alkaline earth metal hydroxides (for example Mg(OH)₂), the alkaline earth metal oxides (for example CaO) or the alkaline earth metal carbonates (for example CaCO₃), ammonia, aliphatic amines (for example, triethylamine or diisopropylamine) or the heterocyclic amines (for example pyridine or 4-(N,N-dimethylamino)pyridine) or the basic inorganic or organic ion exchangers.

Furthermore, the present invention also relates to cosmetic and dermatological preparations which comprise the inventive 2-(3,4-dihydroxyphenyl)ethyl-substituted carbonic acid derivatives in an active amount in addition to other otherwise customary composition constituents. They contain 0.0001% by weight to 10% by weight, preferably 0.001 to 5% by weight, but more preferably, 0.001% by weight to 1% by weight, based on the total weight of the formulation of the inventive 2-(3,4-dihydroxy-phenyl)ethyl-substituted carbonic acid derivatives and can be here “water in oil”, “oil in water”, “water in oil in water”, or “oil in water in oil” emulsions, microemulsions, gels, solutions, for example in oils, alcohols or silicone oils, soaps, sticks, aerosols, sprays or else foams. Other customary cosmetic or dermatological auxiliaries and additives can be present in amounts of 5 to 99.9999% by weight, preferably 10 to 80% by weight, based on the total weight of the formulation. In addition, the formulations can comprise water in an amount up to 99.999% by weight, preferably 5 to 80% by weight, based on the total weight of the formulation.

The present invention, in addition, also relates to preparations serving for nutrition or pleasure which comprise the inventive 2-(3,4-dihydroxyphenyl)ethyl-substituted carbonic acid derivatives in an active amount in addition to other otherwise customary composition constituents. They contain 0.0001% by weight to 10% by weight, preferably 0.001 to 5% by weight, but more preferably, 0.001% by weight to 1% by weight, based on the total weight of the formulation of the inventive 2-(3,4-dihydroxy-phenyl)ethyl-substituted carbonic acid derivatives and they can be gels, solutions, for example in oils, water, ethanol, dispersions, emulsions or suspensions in oil or water, or else mixed with dry compositions. Other customary base substances, auxiliaries and additives serving for nutrition or pleasure can be present in amounts of 5 to 99.9999% by weight, preferably 10 to 80% by weight, based on the total weight of the formulation. In addition, the formulations can comprise water in an amount up to 99.999% by weight, preferably 5 to 80% by weight, based on the total weight of the formulation.

The inventive preparations comprising the 2-(3,4-dihydroxyphenyl)-ethyl-substituted carbonic acid derivatives are prepared by customary processes known per se in such a manner that one or more of the inventive 2-(3,4-dihydroxyphenyl)ethyl-substituted carbonic acid derivatives are incorporated into the formulations which are composed as is customary and can serve for treatment, protection, care and cleaning of the skin or the hair, as make-up products and as foods or drinks.

To prepare the inventive preparations, in a further embodiment, the inventive 2-(3,4-dihydroxyphenyl)ethyl-substituted carbonic acid derivatives can also be incorporated in advance into liposomes, for example starting from phosphatidyl choline, into microspheres, into nanospheres or else into capsules of a suitable matrix, for example natural or synthetic waxes, for examples beeswax, carnauba wax, silicone wax or paraffin waxes, and also stearyl alcohol, eicosanol, cetyl alcohol, stearin, carbohydrates, for example starch, or of proteins, for example gelatins. A further embodiment is that the inventive 2-(3,4-dihydroxyphenyl)ethyl-substituted carbonic acid derivatives are complexed in advance with complexing agents, for example with cyclodextrins or cyclodextrin derivatives, preferably methylcyclodextrin.

The inventive cosmetic and dermatological preparations can comprise cosmetic auxiliaries and additives as are customarily used in such preparations, for example sunscreens (for example organic or inorganic light filtering substances, preferably micropigments), preservatives, bactericides, fungicides, viricides, compounds having a cooling action, plant extracts, anti-inflammatory compounds, substances accelerating wound healing (for example chitin or chitosan and its derivatives), film-forming substances (for example polyvinylpyrrolidones or chitosan or its derivatives), customary antioxidants, vitamins (for example vitamin C and derivatives, tocopherols and derivatives, vitamin A and derivatives), 2-hydroxycarboxylic acids (for example citric acid, malic acid, L-, D- or dl-lactic acid), skin lighteners (for example kojic acid, hydroquinone or arbutin), skin pigmenting agents (for example walnut extracts or dihydroxyacetone), perfumes, substances for preventing foaming, dyes, pigments which have a coloring action, thickeners, surface-active substances, emulsifiers, plasticizing, moisturizing and/or moisture-retaining substances (for example glycerol or urea), fats, oils, unsaturated fatty acids or their derivatives (for example linoleic acid, α-linolenic acid, γ-linolenic acid or arachidonic acid and their respective natural or synthetic esters), waxes or other customary constituents of a cosmetic or dermatological formulation such as alcohols, polyols, polymers, foam stabilizers, electrolytes, organic solvents, silicone derivatives or chelating agents (for example, ethylenediaminetetraacetic acid and derivatives).

The inventive preparations serving for nutrition or pleasure can comprise base substances, auxiliaries and additives, as are customarily used in such preparations, for example meat products, fish products, cereal products, vegetable products, fruit products, carbohydrates, proteins, peptides, amino acids, natural or synthetic sweeteners, mineral salts, bitter substances, mineral or organic acids, taste modulators, preservatives, bactericides, fungicides, viricides, plant extracts, animal extracts, customary antioxidants, vitamines (for example vitamin A, B group vitamins, vitamin C, tocopherol), 2-hydroxycarboxylic acids (for example, citric acid, malic acid, L-, D- or dl-lactic acid), flavors, substances for preventing foaming, dyes, pigments which have a coloring action, thickeners, surface active substances, emulsifiers, fats, oils, unsaturated fatty acids or their derivatives (for example linoleic acid, α-linolenic acid, γ-linolenic acid, eicosapentanoic acid, docosahexanoic acid or arachidonic acid and their respective natural esters), waxes or other customary constituents.

The amounts to be used in each case can, depending on the type of the respective product, be readily determined by those skilled in the art by simple trials.

Preferably, the inventive preparations, in addition to one or more of the inventive 2-(3,4-dihydroxyphenyl)ethyl-substituted carbonic acid derivatives, can also comprise other antioxidants. In particular, the other antioxidants which can be used are all antioxidants which are suitable or customary for the corresponding applications. Advantageously, the antioxidants are selected from the group consisting of amino acids (for example glycine, histidine, 3,4-dihydroxyphenylalanine, tyrosine, tryptophan) and derivatives thereof, imidazoles (for example urocanic acid) and derivatives thereof, peptides (D,L-carnosine, D-carnosine, L-carnosine, anserine) and derivatives thereof, carotenoids, carotenes (for example α-carotene, β-carotene, lycopene) and their derivatives, chlorogenic acids and derivatives thereof, lipoic acid and derivatives thereof, aurothioglucose, propylthiouracil and other thiols (for example thioredoxin, glutathione, cysteine, cystine, cystamine and glycosyl and N-acyl derivatives or alkyl esters thereof) and salts thereof, dilauryl thiodipropionate, distearyl thiodipropionate, thiodipropionic acid and derivatives thereof, and phenolic amides of phenolic benzylamines (for example homovanillic, 3,4-dihydroxyphenylacetic, ferulic, sinapic, caffeic, dihydroferulic, dihydrocaffeic, vanillomandelic or 3,4-dihydroxymandelic amides of 3,4-dihydroxybenzylamine, 2,3,4-trihydroxybenzylamine or 3,4,5-trihydroxybenzylamine), catechol oximes or catechol oxime ethers (for example 3,4-dihydroxybenzaldoxime or 3,4-dihydroxybenzaldehyde O-ethyloxime), in addition (metal) chelators (for example 2-hydroxy fatty acids, phytic acid, lactoferrin), humic acid, bile acids, bile extracts, bilirubin, biliverdin, folic acid and derivatives thereof, ubiquinone and ubiquinol and derivatives thereof, vitamin C and derivatives thereof (for example ascorbyl palmitate, magnesium ascorbyl phosphate, ascorbyl acetate), tocopherols and derivatives (for example vitamin E acetate), vitamin A and derivatives (for example vitamin A palmitate), rutic acid and derivatives thereof, flavonoids (for example quercetin, α-glucosylrutin) and derivatives thereof, phenolic acids (for example gallic acid, ferulic acid) and derivatives thereof (for example propyl, ethyl and octyl esters of gallic acid), furfurylideneglucitol, dibutylhydroxytoluene, butylhydroxyanisole, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, resveratrol) and the derivatives of these said active compounds suitable according to the present invention.

The amount of further antioxidants can be, in the inventive preparations, generally 0.0001 to 30% by weight, preferably 0.001 to 20% by weight, more preferably 0.001 to 5% by weight, based on the total weight of the preparation.

In addition to the inventive 2-(3,4-dihydroxyphenyl)ethyl-substituted carbonic acid derivatives, obviously, a plurality of other antioxidants can be used.

In the inventive cosmetic or dermatological preparations, however, UV-A and/or UV-B filter substances can also be used, in which case the total amount of filter substances can be 0.1 to 30% by weight, preferably 0.5 to 10% by weight, based on the total weight of the preparations, sunscreens for skin and hair, for example, being obtained. UV-A and/or UV-B filter substances which can be used are, for example, 3-benzylidenecamphor derivatives, (for example 3-(4-methyl-benzylidene)-dl-camphor), aminobenzoic acid derivatives (for example 2-ethylhexyl 4-(N,N-dimethylamino)benzoate or menthyl anthranilate), 4-methoxycinnamates (for example 2-ethylhexyl p-methoxycinnamate or isoamyl p-methoxycinnamate), benzophenones (for example 2-hydroxy-4-methoxybenzophenone), mono-sulfonated or polysulfonated UV filters [for example 2-phenylbenzimidazole-5-sulphonic acid, sulisobenzones or 1,4-bis(benzimidazolyl)benzene-4,4′,6,6′-tetrasulphonic acid and 3,3′-(1,4-phenylenedimethylidenee)bis-(7,7-dimethyl-2-oxo-bicyclo-[2,2,1]heptane-1-methanesulphonic acid) and salts thereof], salicylates (for example 2-ethylhexylsalicylate or homomenthyl salicylate), triazines {for example 2,4-bis[4-(2-ethylhexyloxy)-2-hydroxyphenyl]-6-(4-methoxyphenyl)-1,3,5-triazine, bis-(2-ethylhexyl) 4,4′-([6-([(1,1-dimethyl-ethyl)aminoca rbonyl]phenylamino)-1,3,5-triazine-2,4-diyl]diimino)bisbenzoate}, 2-cyanopropenoic acid derivatives (for example 2-ethylhexyl 2-cyano-3,3-diphenyl-2-propenoate), dibenzoyl derivatives (for example 4-tert-butyl-4′-methoxydibenzoylmethane), polymer-bound UV filters (for example polymers of N-[2-(or 4)-(2-oxo-3-bornylidene)methyl]benzyl-acrylamide) or pigments (for example titanium dioxides, zirconium dioxides, iron oxides, silicon dioxides, manganese oxides, aluminum oxides, cerium oxides or zinc oxides).

The lipid phase in the inventive cosmetic and/or dermatological preparations can advantageously be selected from the following groups of substances: mineral oils (advantageously paraffin oil), mineral waxes, hydrocarbons (advantageously squalane or squalene), synthetic or semisynthetic triglyceride oils (for example triglycerides of capric or caprylic acid), natural oils (for example castor oil, olive oil, sunflower oil, soya bean oil, peanut oil, rapeseed oil, almond oil, palm oil, coconut oil, palm kernel oil, borage seed oil, and others of the like), natural ester oils (for example jojoba oil), synthetic ester oils (preferably esters of saturated and/or unsaturated unbranched and/or branched alkanecarboxylic acids of 3 to 30 carbon atoms with saturated and/or unsaturated, unbranched and/or branched alcohols having 3 to 30 carbon atoms and esters of aromatic carboxylic acids with saturated and/or unsaturated, unbranched and/or branched alcohols having 3 to 30 carbon atoms, in particular selected from the group containing isopropyl myristate, isopropyl stearate, isopropyl palmitate, isopropyl oleate, n-butyl stearate, n-hexyl laurate, n-decyl laurate, isooctyl stearate, isononyl stearate, isononyl isononanoate, 2-ethylhexyl palmitate, 2-ethylhexyl laureate, 2-hexyldecyl stearate, 2-octyldecyl palmitate, oleyl oleate, oleyl erucate, erucyl oleate, erucyl erucate and synthetic or natural mixtures of such esters), fats, waxes and other natural and synthetic lipids, preferably esters of fatty alcohols with alcohols of low carbon number (for example with isopropanol, propylene glycol or glycerol) or esters of fatty alcohols with alkanoic acids with low carbon number or with fatty acids, alkylbenzoates (for example mixtures of n-dodecyl, n-tridecyl, n-tetradecyl and n-pentadecyl benzoate) and cyclic or linear silicone oils (for example dimethyl-polysiloxanes, diethylpolysiloxanes, diphenylpolysiloxanes and mixed forms thereof).

The aqueous phase of the inventive cosmetic and/or dermatological preparations may advantageously comprise alcohols, diols or polyols of low carbon number, and ethers thereof, preferably ethanol, isopropanol, propylene glycol, glycerol, ethylene glycol, ethylene glycol monoethyl ether or ethylene glycol monobutyl ether, propylene glycol monomethyl ether, propylene glycol monoethyl ether or propylene glycol monobutyl ether, diethylene glycol monomethyl ether or diethylene glycol monoethyl ether and similar products, in addition alcohols of low carbon number, for example ethanol, isopropanol, 1,2-propanediol, glycerol, in addition α- or β-hydroxyacids, preferably lactic acid, citric acid or salicylic acid, in addition emulsifiers, which can advantageously be selected from the group consisting of ionic, nonionic, polymeric, phosphate-containing and zwitterionic emulsifiers, and, in particular, one or more thickeners, which can advantageously be selected from the group silicon dioxide, aluminum silicates, for example bentonites, polysaccharides and derivatives thereof, for example hyaluronic acid, guar bean meal, xanthan gum, hydroxypropyl methyl cellulose or allulose derivatives, particularly advantageously from the group of the polyacrylates, preferably a polyacrylate from the group of the carbopols, in each case individually or in combination, or from the group of the polyurethanes.

The inventive preparations serving for nutrition or pleasure can, in addition to customarily used animal or plant raw materials, additionally comprise water, squalane or squalene, natural oils (for example olive oil, sunflower oil, soya bean oil, peanut oil, rapeseed oil, almond oil, palm oil, coconut oil, palm kernel oil, borage seed oil and more of the like), natural ester oils (for example jojoba oil), fats, waxes and other natural lipids, carbohydrates, for example glucose, sucrose or lactose, sweeteners, for example aspartame, cyclamate, saccharin, xylitol or sorbitol, bitter substances, for example caffeine or quinine, bitterness-suppressing substances, for example Lactisol, taste-enhancing substances, for example sodium glutamate or inositol phosphate, amino acids, for example glycine, alanine, leucine, isoleucine, valine, proline, lysine, asparagine, aspartic acid, glutamine, glutamic acid, tryptophan, phenylalanine, tyrosine, threonin, serine, cystine, cysteine, methionine, hydroxyproline, arginine or histidine, peptides, proteins, enzymes, fruit acids, preferably lactic acid, malic acid or citric acid, in addition emulsifiers, which advantageously can be selected from the group of the ionic, nonionic, polymeric, phosphate-containing and zwitterionic emulsifiers, and, in particular, one or more thickeners, which can advantageously be selected from the group of the polysaccharides or derivatives thereof, for example hyaluronic acid, guar bean meal, carob bean meal, xanthan gum, or allulose derivatives, natural, nature-identical or synthetic flavors, and salts, for example sodium chloride or potassium chloride.

An advantageous embodiment of the present invention is considered to be the use of the inventive preparations comprising an active component of the inventive 2-(3,4-dihydroxyphenyl)ethyl-substituted carbonic acid derivatives for the protection of tissues and cells of mammals against oxidative stress and the harmful effect of free radicals.

The present invention also contains a process for protecting cosmetic preparations, dermatological preparations and preparations serving for nutrition or pleasure against oxidation or photooxidation, in which case these preparations can be, for example, preparations for treatment, protection and care of the skin, nails or hair or else foods or drinks, whose constituents are accompanied by stability problems owing to oxidation or photooxidation during storage, characterized in that the inventive preparations have an active content of inventive 2-(3,4-dihydroxy-phenyl)ethyl-substituted carbonic acid derivatives.

### EXAMPLES

The examples below are intended to explain the present invention without restricting it.

Synthesis Protocols

### Example 1

### N-[2-(3,4-Dihydroxyphenyl)ethyl]-O-methylurethane)

2-(3,4-Dihydroxyphenyl)ethylamine hydrochloride (3 g, 15.9 mmol) is dissolved in 1,4-dioxane and water (1:1, 60 ml) and sodium hydrogencarbonate (1.33 g) is added. Methyl chloroformate (1.5 g, 15.9 mmol) is added dropwise to the mixture. After adding half of the solution, a portion of sodium hydrogencarbonate (1.33 g, in total 15.9 mmol) is again added. The mixture is further stirred for 2 h at room temperature, brought to pH 1-2 using 5% strength H₂SO₄, extracted with tert-butyl methyl ether (3 times 50 ml), the combined organic phases are washed with saturated NaCl solution, dried over Na₂SO₄, filtered and the filtrate is evaporated at 45° C./20 mbar (5.7 g, yellow oil). The crude product is dissolved in 1,4-dioxane and water (1:1, 20 ml), saturated NaHCO₃ solution (1 ml) is added and the mixture is stirred for 3 h at 80° C. The reaction mixture is brought to approximately pH 4 using 5% strength H₂SO₄, extracted with tert-butyl methyl ether (3 times 50 ml), the combined organic phases are washed with saturated NaCl solution, dried over Na₂SO₄, filtered and the filtrate is evaporated at 45° C./20 mbar (5.5 g). The product is purified on silica gel 60 using the eluents CHCl₃/CH₃OH 7:1 (v/v): 3.3 g of yellow oil (99% of theory); purity 99% (HPLC); ¹H-NMR (400 MHz, CD₃OD): δ=6.67 (1H, d, 8 Hz), 6.62 (1H, d, 2 Hz), 6.51 (1H, dd, 8 Hz, 2 Hz), 3.62 (3H, s), 3.24 (2H, t, 7 Hz), 2.62 (2H, t, 7 Hz) ppm; MS (APCl−): m/z=210.35 (47%, [M−H]⁻), 420.75 (100%, [2M−H]⁻).

The following compounds were obtained in a similar manner:

### Example 2

### N-[2-(3,4-Dihydroxyphenyl)ethyl]-O-[(1R,3R,4S)-menthyl]urethane

Yield: quantitative; purity 100% (HPLC); ¹H-NMR (200 MHz, CD₃OD): δ=6.66 (1H, d, 7.5 Hz), 6.62 (1H, d, 2 Hz), 6.49 (1H, dd, 7.5 Hz, 2 Hz), 4.47 (1H, td, 11 Hz, 5 Hz), 3.23 (2H, t, 7 Hz), 2.60 (2H, t, 7 Hz), 2.03-1.83 (2H, m), 1.78-1.60 (2H, m), 1.6-0.7 (m, 14H) ppm; MS (ESI+): m/z=198.80 (100%), 336.45 (96%, [M+H]⁺).

### Example 3

### N-[2-(3,4-Dihydroxyphenyl)ethyl]-O-hexylurethane

Yield 55%; ¹H-NMR (400 MHz, CD₃OD): δ=6.67 (1H, d, 8 Hz), 6.63 (1H, d, 2 Hz), 6.50 (1H, dd, 8 Hz, 2 Hz), 3.99 (2H, t, 7 Hz), 3.24 (2H, t, 7.5 Hz), 2.62 (2H, t, 7.5 Hz), 1.58 (2H, m), 1.42-1.25 (6H, m), 0.91 (3H, t, 7 Hz) ppm; purity: 99%; MS (APCl+): m/z=281.99 (100%, [M+H]⁺).

### Example 4

### N-[2-(3,4-Dihydroxyphenyl)ethyl]-O-(2-ethylhexyl)urethane

Yield 80%; purity 100% (HPLC); ¹H-NMR (400 MHz, CD₃OD): δ=6.68 (1H, d, 8 Hz), 6.62 (1H, d, 2 Hz), 6.50 (1H, dd, (Hz, 2 Hz), 3.93 (2H, d, 7 Hz), 3.23 (2H, t, 7.5 Hz), 2.61 (2H, t, 7.5 Hz), 1.54 (1H, m), 1.42-1.26 (8H, m), 0.96-0.84 (6H, m) ppm; MS (APCl−): m/z=308.27 (32%, [M−H]⁻), 350.70 (100%).

### Example 5

### N-[2-(3,4-Dihydroxyphenyl)ethyl]-N′-hexylthiourea

2-(3,4-Dihydroxyphenyl)ethylamine hydrochloride (0.99 g, 5 mmol) and triethylamine (1.01 g, 10 mmol) are suspended in CHCl₃ (15 ml) under nitrogen and n-hexyl isothiocyanate (0.75 g, 5.25 mmol) dissolved in CHCl₃ (10 ml) is added. The reaction mixture is stirred for a further 18 h at room temperature and then washed with 5% strength hydrochloric acid. The organic phase is washed with water, dried over Na₂SO₄, filtered and the filtrate is evaporated at 40° C./800-20 mbar: 0.795 g (54%); MS (APCl−): m/z=295.59 (100%, [M−H]⁻), 590.94 (4%, [2M−H]⁻).

### Example 6

### N-[2-(3,4-Dihydroxyphenyl)ethyl]-N′-hexylurea

2-(3,4-Dihydroxyphenyl)ethylamine hydrochloride (1 g, 5.3 mmol) and triethylamine (2.04 g, 20 mmol) are suspended in CHCl₃ (25 ml) under nitrogen and n-hexyl isocyanate (0.51 g, 4.06 mmol) dissolved in CHCl₃ (20 ml) is added in the course of 20 min. The reaction mixture is stirred for a further 18 h at room temperature and then washed with 5% strength hydrochloric acid. The organic phase is washed with water, dried over Na₂SO₄, filtered and the filtrate is evaporated at 40° C./800-20 mbar (1.5 g); the filtrate is chromatographed on silica gel using chloroform/methanol 10:1. Yield 0.38 g (26%); ¹H-NMR (400 MHz, CD₃OD): δ=6.69 (1H, d, 8 Hz), 6.64 (1H, d, 2 Hz), 6.52 (1H, dd, 8 Hz, 2 Hz), 3.26 (2H, t, 7 Hz), 3.07 (2H, t, 7 Hz), 2.62 (2H, t, 7 Hz), 1.45 (2H, m), 1.38-1.25 (8H, m), 0.90 (3H, t, 7 Hz) ppm; MS (APCl+): m/z=281.30 (100%, [M+H]⁺), 560.92 (77%, [2M+H]⁺).

### Application Examples:

### Example 7

### Cosmetic “Oil in Water” Emulsion

TABLE 1

| | | | Content |
|---|---|---|---|
| | Raw Material Name | | in % by |
| Part | (Manufacturer) | Chemical Name | weight |
| A | Arlatone 983 S ® (ICI) | Ether of polyethylene glycol | 1.2 |
| | | with glyceryl monostearate | |
| | Brij 76 ® (ICI) | 3,6,9,12,15,18,21,24,27,30, | |
| | | 33,36-decaoxaoctate- | 1.2 |
| | | tracontan-1-ol | |
| | Cutina MD ® (Henkel) | Glyceryl monostearate | 3.5 |
| | Baysilone oil M10 ® | Polydimethylsiloxane | 0.8 |
| | (GE Bayer) | | |
| | Eutanol G ® (Henkel) | Octyldodecanol | 3.0 |
| | Paraffin oil 65 cp | Mineral oil | 8.0 |
| | (Henry Lamotte) | | |
| B | Water, distilled | | 50.35 |
| | Phenonip ® | 2-Phenoxyethanol and | 0.5 |
| | (Nipa Laboratories) | methyl 4-hydroxybenzoate | |
| | | and ethyl 4-hydroxybenzoate | |
| | | and propyl | |
| | | 4-hydroxybenzoate and butyl | |
| | | 4-hydroxybenzoate | |
| | 1,2-Propylene glycol | | 2.0 |
| | Glycerol 99% | | 3.0 |
| | Trilon ® BD | Disodium EDTA | 0.1 |
| | N-[2-(3,4- | | 0.1 |
| | Dihydroxyphenyl)ethyl]- | | |
| | O-methylurethane | | |
| | (Example 1) | | |
| C | Water, distilled | | 25.0 |
| | Carbopol 2050 ® (B. F. | Crosslinked acrylic acid/ | 0.4 |
| | Goodrich) | C₁₀—C₃₀-alkyl acrylate | |
| | | polymer | |
| | Aqueous sodium | | 0.85 |
| | hydroxide solution, 10% | | |

Part A was mixed and heated to 80° C. Part B was mixed and heated to 90° C. and added to part A with stirring. For part C, Carbopol was carefully dispersed in water and neutralized with sodium hydroxide solution (pH 5.4). Part C was then added at 60° C. to the mixture of parts A and B.

### Example 8

### Cosmetic Butylene Glycol Solution

TABLE 2

| | Content |
|---|---|
| | in % by |
| Raw Material Name | weight |
| 1,3-Butylene glycol | 99.9 |
| N-[2-(3,4-Dihydroxyphenyl)ethyl]-O- | 0.1 |
| methylurethane (Example 1) | |

### Example 9

### Cosmetic “Water in Oil” Sunscreen Emulsion Having UVA/B Broadband Protection

TABLE 3

| | | | Content |
|---|---|---|---|
| | Raw Material Name | | in % by |
| Part | (Manufacturer) | Chemical Name | weight |
| A | Dehymuls PGPH ® | Polyglyceryl-2 | 3.0 |
| | (Henkel) | dipolyhydroxystearate | |
| | Monomuls 90-O 18 ® | Glyceryl oleate | 1.0 |
| | (Henkel) | | |
| | Permulgin 2550 ® | Beeswax | 1.0 |
| | (Koster Keunen Holland) | | |
| | Myritol 318 ® (Henkel) | Caprylic/caproic | 6.0 |
| | | triglycerides | |
| | Witconol TN ® (Witco) | C₁₂—C₁₅-Alkyl benzoate | 6.0 |
| | Cetiol SN ® (Henkel) | Cetyl and stearyl | 5.0 |
| | | isononanoate | |
| | Copherol 1250 ® | Tocopherol acetate | 1.0 |
| | (Henkel) | | |
| | Solbrol P ® (Bayer) | Propyl 4-hydroxybenzoate | 0.1 |
| | Neo Heliopan ® AV | 2-Ethylhexyl p-methoxy- | 4.0 |
| | (Haarmann & Reimer) | cinnamate | |
| | Neo Heliopan ® E 1000 | Isoamyl p- | |
| | (Haarmann & Reimer) | methoxycinnamate | 4.0 |
| | Neo Heliopan ® MBC | 3-(4-Methylbenzylidene)-dl- | 2.0 |
| | (Haarmann & Reimer) | camphor | |
| | Neo Heliopan ® OS | 2-Ethylhexyl salicylate | 3.0 |
| | (Haarmann & Reimer) | | |
| | Octyltriazone | | 1.0 |
| | Zinc oxide neutral | | 7.0 |
| | (Haarmann & Reimer) | | |
| B | Water, distilled | | 40 |
| | Phenoxyethanol | | 0.7 |
| | Solbrol ® M (Bayer) | Methyl 4-hydroxybenzoate | 0.2 |
| | Glycerol 99% | | 4.0 |
| | Neo Heliopan ® Hydro | 2-Phenylbenzimidazole-5- | 10.0 |
| | (Haarmann & Reimer), | sulphonic acid | |
| | 15% as sodium salt | | |
| | 4-Benzophenone | | 0.5 |
| | N-[2-(3,4- | | 0.1 |
| | Dihydroxyphenyl)ethyl]- | | |
| | O-methylurethane | | |
| | (Example 1) | | |
| C | Perfume oil | | 0.3 |
| | Bisabolol | | 0.1 |

For part A, all substances except the zinc oxide were heated to 85° C. and the zinc oxide was carefully dispersed in the mixture. The components of part B were mixed, heated to 85° C. and added to part A with stirring. Part C was added to the mixture of parts A and B and then the mixture was homogenized using a dispersion appliance.

### Example 10

### Preparation of an Edible Oil for Deep-Frying

TABLE 4

| | Content |
|---|---|
| | in % by |
| Raw Material Name | weight |
| Soya bean oil, refined | 99.95 |
| N-[2-(3,4-Dihydroxyphenyl)ethyl]-N′-hexylurea | 0.05 |
| (Example 6) | |

Experiments

### Example 11

### Activity as Free-Radical Scavenger

The activity of the exemplary compounds as free-radical scavengers was compared with conventional free-radical scavengers. For this, the DPPH (1,1-diphenyl-2-picryl-hydrazyl) test for the elimination of free-radicals was employed (Lebensm.-Wiss. u. Technol., 1995, vol. 28, pp. 25 to 30).

DPPH was dissolved in methanol to give a concentration of 100 μmol/l. A series of dilutions of the example compounds, vitamin C, α-tocopherol and 3,5-ditert-butyl-4-hydroxytoluene were prepared in methanol. Methanol served as control. 2500 μl of the DPPH solution were mixed with 500 μl of each test solution and the decrease in absorption at 515 nm was read off until the decrease was less than 2% per hour. The activity of the test substances as free-radical scavengers was calculated from the following equation:

Activity as free-radical scavenger (%)=100−(absorption of the test compounds)/absorption of the control)×100.

From activity as free-radical scavenger (%) in a number of dilutions of test compounds, for each test compound the effective relative concentration EC₅₀ (based on the initial concentration of DPPH, EC=c (test compound)/c(DPPH)) of a test compound was calculated at which the free-radical DPPH was eliminated by 50%. The smaller the EC₅₀ values, the better is the free-radical scavenging action.

The results are shown in Table 5:

TABLE 5

| | EC₅₀/ |
|---|---|
| Test Compound (example number) | (mol/mol) |
| N-[2-(3,4-Dihydroxyphenyl)ethyl]-O-methylurethane (1) | 0.27 |
| N-[2-(3,4-Dihydroxyphenyl)ethyl]-O-[(1R,3R,4S)-menthyl] | 0.35 |
| urethane (2) | |
| N-[2-(3,4-Dihydroxyphenyl)ethyl]-O-hexylurethane (3) | 0.24 |
| N-[2-(3,4-Dihydroxyphenyl)ethyl]-O-(2-ethylhexyl)urethane | 0.25 |
| (4) | |
| N-[2-(3,4-Dihydroxyphenyl)ethyl]-N′-hexylurea (6) | 0.20 |
| Vitamin C | 0.27 |
| α-Tocopherol | 0.25 |
| 3,5-Ditert-butyl-4-hydroxytoluene | 0.24 |

### Example 12

### Activity as Antioxidants

The activity of the example compounds as antioxidants was compared with that of conventional antioxidants. The test system used was accelerated autoxidation of lipids by air with or without antioxidants, using the Rancimat apparatus (Deutsche Lebensmiftel-Rundschau 1974, vol. 70, pp. 57 to 65) (Rancimat is a registered trademark of Metrohm AG, Herisau, Switzerland).

The example compounds, vitamin C, α-tocopherol and 3,5-ditert-butyl-4-hydroxytoluene were dissolved in methanol or acetone and 100 μl of the respective test solution were added to a prepared oil sample of 3 g. Only solvent was added to a control sample. A constant dry air stream (20 I/h) was blown through the heated oil sample containing the test solution and the volatile oxidation products (principally short-chain fatty acids, such as formic acid or acetic acid) were collected in a receiver containing water. The conductivity of this aqueous solution was measured continuously and documented. The oxidation of (unsaturated) fats proceeds here only very slowly for a period, and then suddenly increases. The time until the increase is termed the induction period (IP).

From the equation below, the antioxidant power (AP) was obtained:

AP=IP_{(with test solution)}/IP_{(control sample).}

The higher the AP, the higher is the antioxidant activity.

The results of the experiment at 100° C. in soya bean oil which was purified on alumina type N are shown in Table 6:

TABLE 6

| | AP in soya |
|---|---|
| | bean oil at |
| | 100° C. |
| | containing |
| | 0.05% of test |
| Test Compound (example number) | substance |
| N-[2-(3,4-Dihydroxyphenyl)ethyl]-O-methylurethane (1) | 15.3 |
| N-[2-(3,4-Dihydroxyphenyl)ethyl]-O-[(1R,3R,4S)- | 7.1 |
| menthyl]urethane (2) | |
| N-[2-(3,4-Dihydroxyphenyl)ethyl]-O-hexylurethane (3) | 9.8 |
| N-[2-(3,4-Dihydroxyphenyl)ethyl]-O-(2-ethylhexyl) | 7.6 |
| urethane (4) | |
| N-[2-(3,4-Dihydroxyphenyl)ethyl]-N′-hexylthiourea (5) | 14.4 |
| N-[2-(3,4-Dihydroxyphenyl)ethyl]-N′-hexylurea (6) | 26 |
| Vitamin C | 1.2 |
| α-Tocopherol | 5.1 |
| 3,5-Ditert-butyl-4-hydroxytoluene | 4.8 |

The results for the experiment at 80° C. in squalene which was purified on alumina type N and stabilized with 1 ppm of α-tocopherol are shown in Table 7:

TABLE 7

| | AP in |
|---|---|
| | squalene at |
| | 80° C. |
| | containing |
| | 0.005% test |
| Test Compound (example number) | substance |
| N-[2-(3,4-Dihydroxyphenyl)ethyl]-O-methylurethane (1) | 76 |
| N-[2-(3,4-Dihydroxyphenyl)ethyl]-O-[(1R,3R,4S)- | 25 |
| menthyl]urethane (2) | |
| N-[2-(3,4-Dihydroxyphenyl)ethyl]-O-hexylurethane (3) | 46 |
| N-[2-(3,4-Dihydroxyphenyl)ethyl]-O-(2-ethylhexyl) | 50 |
| urethane (4) | |
| N-[2-(3,4-Dihydroxyphenyl)ethyl]-N′-hexylthiourea (5) | 59 |
| N-[2-(3,4-Dihydroxyphenyl)ethyl]-N′-hexylurea (6) | 60 |
| Vitamin C | 0.7 |
| α-Tocopherol | 39 |
| 3,5-Ditert-butyl-4-hydroxytoluene | 38 |

As can be seen from the experiments in Examples 11 and 12, the inventive compounds are equally good free-radical scavengers, and even significantly better antioxidants, compared with the standard antioxidants vitamin C, α-tocopherol or 3,5-ditert-butyl-4-hydroxytoluene.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. An antioxidant or free-radical scavenger of 2-(3,4-dihydroxyphenyl)ethyl-substituted carbonic acid derivatives of the general formula I 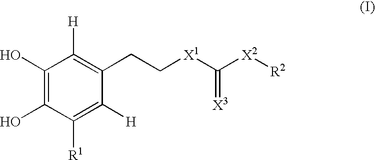
wherein
R¹ is a hydrogen atom, a lower alkyl, lower alkenyl, 1-oxo lower alkyl or 1-oxo lower alkenyl group or a group —O—R³, where R³ is a hydrogen atom, a lower alkyl, lower alkenyl, 1-oxo lower alkyl or 1-oxo lower alkenyl group, and
X¹, X² and X³ independently of one another are oxygen, sulfur or NH, and
R² is a branched or unbranched cyclic or extended alkyl group having 1 to 22 carbon atoms or a branched or unbranched cyclic or extended alkenyl group having 2 to 22 carbon atoms, in which one or more of the carbon atoms can be replaced by oxygen atoms, or a nuclear-substituted arylalkyl group having 7 to 15 carbon atoms.

2. An antioxidant or free-radical scavenger according to claim 1, wherein
R¹ is a hydrogen atom, a lower alkyl group having 1 to 5 carbon atoms or a group —O—R³, where R³ is a hydrogen atom or a methyl group, and
X¹, X² and X³ independently of one another are oxygen, sulfur or NH, and
R² is a branched or unbranched, cyclic or extended alkyl group having 1 to 18 carbon atoms or a branched or unbranched, cyclic or extended alkenyl group having 2 to 20 carbon atoms, or a nuclear-substituted benzyl, nuclear-substituted 2-phenylethyl or nuclear-substituted 1-phenylethyl radical.

3. An antioxidant or free-radical scavenger according to claim 1, wherein said antioxidant or free-radical scavenger is N-[2-(3,4-dihydroxy-phenyl)ethyl]-O-methylurethane, N-[2-(3,4-dihydroxy-phenyl)ethyl]-O-[(1R,3R,4S)-menthyl]urethane, N-[2-(3,4-dihydroxy-phenyl)ethyl]-O-hexylurethane, N-[2-(3,4-dihydroxyphenyl)ethyl]-O-(2-ethylhexyl)urethane, N-[2-(3,4-dihydroxyphenyl)ethyl]-N′-hexylthiourea or N-[2-(3,4-dihydroxyphenyl)ethyl]-N′-hexylurea.

4. Cosmetic preparations, dermatological preparations and preparations serving for nutrition or pleasure comprising 2-(3,4-dihydroxy-phenyl)ethyl-substituted carbonic acid derivatives of the general formula I 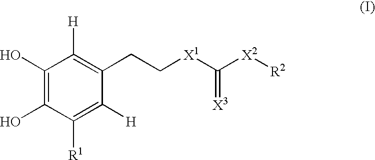
wherein
R¹ is a hydrogen atom, a lower alkyl, lower alkenyl, 1-oxo lower alkyl or 1-oxo lower alkenyl group or a group —O—R³, where R³ is a hydrogen atom, a lower alkyl, lower alkenyl, 1-oxo lower alkyl or 1-oxo lower alkenyl group, and
X¹, X² and X³ independently of one another are oxygen, sulfur or NH, and
R² is a branched or unbranched cyclic or extended alkyl group having 1 to 22 carbon atoms or a branched or unbranched cyclic or extended alkenyl group having 2 to 22 carbon atoms, in which one or more of the carbon atoms can be replaced by oxygen atoms, or a nuclear-substituted arylalkyl group having 7 to 15 carbon atoms.

5. Cosmetic preparations, dermatological preparations and preparations serving for nutrition or pleasure according to claim 4, wherein
R¹ is a hydrogen atom, a lower alkyl group having 1 to 5 carbon atoms or a group —O—R³, where R³ is a hydrogen atom or a methyl group, and
X¹, X² and X³ independently of one another are oxygen, sulfur or NH, and
R² is a branched or unbranched, cyclic or extended alkyl group having 1 to 18 carbon atoms or a branched or unbranched, cyclic or extended alkenyl group having 2 to 20 carbon atoms, or a nuclear-substituted benzyl, nuclear-substituted 2-phenylethyl or nuclear-substituted 1-phenylethyl radical.

6. Cosmetic preparations, dermatological preparations and preparations serving for nutrition or pleasure according to claim 1, wherein said antioxidant or free-radical scavenger is N-[2-(3,4-dihydroxy-phenyl)ethyl]-O-methyl-urethane, N-[2-(3,4-dihydroxyphenyl)ethyl]-O-[(1R,3R,4S)-menthyl]urethane, N-[2-(3,4-dihydroxyphenyl)ethyl]-O-hexylurethane, N-[2-(3,4-dihydroxyphenyl)ethyl]-O-(2-ethylhexyl)urethane, N-[2-(3,4-dihydroxyphenyl)ethyl]-N′-hexylthiourea or N-[2-(3,4-dihydroxyphenyl)ethyl]-N′-hexylurea.

7. 2-(3,4-Dihydroxyphenyl)ethyl-substituted carbonic acid derivatives of the general formula (II) 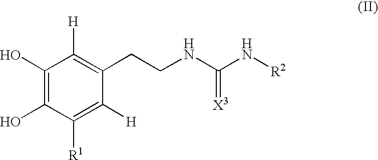
wherein
R¹ is a hydrogen atom, a lower alkyl, lower alkenyl, 1-oxo lower alkyl or 1-oxo lower alkenyl group or a group —O—R³, where R³ is a hydrogen atom, a lower alkyl, lower alkenyl, 1-oxo lower alkyl or 1-oxo lower alkenyl group, and
X³ is oxygen, sulfur or NH, and
R² is a branched or unbranched cyclic or extended alkyl group having 1 to 22 carbon atoms or a branched or unbranched cyclic or extended alkenyl group having 2 to 22 carbon atoms.

8. 2-(3,4-Dihydroxyphenyl)ethyl-substituted carbonic acid derivatives according to claim 7.
wherein
R¹ is a hydrogen atom, a lower alkyl group having 1 to 5 carbon atoms or a group —O—R³, where R³ is a hydrogen atom or a methyl group, and
X³ is oxygen or sulfur, and
R² is a branched or unbranched, cyclic or extended alkyl group having 1 to 18 carbon atoms or a branched or unbranched, cyclic or extended alkenyl group having 2 to 20 carbon atoms.

9. A process for preparing 2-(3,4-dihydroxyphenyl)ethyl substituted carbonic acid derivatives, comprising the step of reacting 2-(3,4-dihydroxyphenyl)ethylamines of the general formula (III) 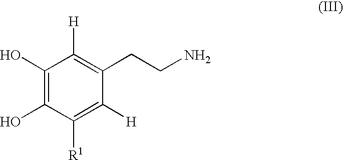
wherein R¹is a hydrogen atom, a lower alkyl, lower alkenyl, 1-oxo lower alkyl or 1-oxo lower alkenyl group or a group —O—R³, where R³ is a hydrogen atom, a lower alkyl, lower alkenyl, 1-oxo lower alkyl or 1-oxo lower alkenyl group,
or cationic salts thereof,
with a heterocumulene of the general formula (IV),
X⁴═C═N—R² (IV)
wherein
X⁴ is an oxygen atom or a sulfur atom and
R² is a branched or unbranched cyclic or extended alkyl group having 1 to 22 carbon atoms or a branched or unbranched cyclic or extended alkenyl group having 2 to 22 carbon atoms, or
with phosgene, thiophosgene or triphosgene with optionally, solvent and with optionally, addition of an auxiliary base and then either directly after purification or without purification with a compound of the general formula (V),
Z-R² (V)
wherein
Z is a group —O—H, —S—H, —NH₂ or —(NH₃)⁺ and
R² has the meaning specified above
with optionally, solvent and with optionally, the addition of an auxiliary base.
